(19) United States
(12) Patent Application Publication
(10) Pub. No.: US 2018/0206729 A1
(43) Pub. Date: Jul. 26, 2018

(54) Wearable patch comprising three electrodes for measurement and charging

(71) Applicant: VivaLnk, Inc., Santa Clara, CA (US)

(72) Inventors: Zhigang Wang, Fremont, CA (US); Jiang Li, Cupertino, CA (US)

(21) Appl. No.: 15/423,585

(22) Filed: Feb. 3, 2017

Related U.S. Application Data
(63) Continuation-in-part of application No. 15/414,549, filed on Jan. 24, 2017.

(51) Int. Cl. A61B 5/01; A61B 5/00; H02J 7/00

(52) U.S. Cl. CPC A61B 5/01; A61B 5/6832; A61B 5/0008; A61B 2562/0276; H02J 7/0054; A61B 2560/0214; A61B 2562/164; H02J 7/0045

(57) ABSTRACT

A wearable patch includes a stretchable and permeable substrate, a temperature sensing unit mounted in the stretchable and permeable substrate, wherein the temperature sensing unit includes a first electrode configured to contact a user's skin, and a temperature sensor in thermal contact with the temperature sensor and configured to measure the user's skin temperature. The wearable patch includes a second electrode and a third electrode respectively attached to the stretchable and permeable substrate, a circuit substrate on the stretchable and permeable substrate, and a battery configured to supply power to the control circuit and the temperature sensor. The control circuit can select a first mode for measuring a user's skin temperature and a second mode for charging the battery by the charging station via the first electrode, the second electrode, and the third electrode.

### CROSS-REFERENCE TO RELATED APPLICATIONS

The present application is also related to commonly assigned pending U.S. patent application Ser. No. 29/562,296, titled “Wearable thermometer having three electric pads”, filed by the same inventors on Apr. 25, 2016, and commonly assigned pending U.S. patent application Ser. No. 29/562,367, titled “Wearable thermometer having three electric pads”, filed by the same inventors on Apr. 25, 2016. The disclosures of the above applications are incorporated herein by reference.

### BACKGROUND OF THE INVENTION

The present application relates to electronic devices, and in particular, to wearable patches that can attach to human skin for conducting measurement.

Electronic patches can be used for tracking objects and for performing functions such as producing sound, light or vibrations, and so on. As applications and human needs become more sophisticated and complex, electronic patches are required to perform a rapidly increasing number of tasks. Electronic patches are often required to be conformal to curved surfaces, which in the case of human body, can vary overtime.

Electronic patches can communicate with smart phones and other devices using WiFi, Bluetooth, Near Field Communication (NFC), and other wireless technologies. NFC is a wireless communication standard that enables two devices to quickly establish communication within a short range around radio frequency of 13.56 MHz. NFC is more secure than other wireless technologies such as Bluetooth and Wi-Fi because NFC requires two devices in close proximity (e.g. less than 10 cm). NFC can also lower cost comparing to other wireless technologies by allowing one of the two devices to be passive (a passive NFC tag).

Bluetooth is another wireless communication standard for exchanging data over relatively longer distances (in tens of meters). It employs short wavelength UHF radio waves from 2.4 to 2.485 GHz from fixed or mobile devices. Bluetooth devices have evolved to meet the increasing demand for low-power solutions that is required for wearable electronics. Benefited from relatively longer reading distance and active communication, Bluetooth technologies allow wearable patches to continuously monitoring vital information without human interference, which is an advantage over NFC in many applications.

Wearable patch (or tag) is an electronic patch to be worn by a user. A wearable patch is required to stay on user's skin and operate for an extended period of time from hours to months. A wearable patch can contain a micro-electronic system that can be accessed using NFC, Bluetooth, WiFi, or other wireless technologies. A wearable patch can be integrated with different sensors such as vital signs monitoring, motion track, skin temperature measurements, and ECG detection.

Despite recent development efforts, current wearable patches still suffer several drawbacks: they may not provide adequate comfort for users to wear them; they may not stay attached to user's body for the required length of time; and they are usually not aesthetically appealing. The conventional wearable patches also include rigid polymer substrates that are not very breathable. The build-up of sweat and moisture can cause discomfort and irritation to the skin, especially after wearing it for an extended period of time.

Conventional wearable thermometer patches have the additional challenge of inaccurate temperature measurement due to factors such as thermal resistance between the temperature sensor and the human skin, conduction loss of the temperature sensor to the ambient environment, as well as temperature reduction in the user skin caused by the thermal conduction to the wearable patch. Moreover, conventional wearable thermometer patches can also have slow measurement responses.

Another drawback of some conventional wearable patches is that they contain batteries that are charged by USB cable. This charging method is inconvenient when the user is not near a power source such as a power outlet or laptop.

Moreover, come conventional wearable patches have limited ranges of wireless communications because their compact sizes usually put constraints on the capacity of the batteries and amplifiers on board.

There is therefore a need for a flexible wearable electronic patch that can correctly measure temperatures of user's skin with high accuracy and fast response time, while capable of performing wireless communications in a required range.

### SUMMARY OF THE INVENTION

The presently disclosure attempts to address the aforementioned limitations in conventional electronic patches. The presently disclosed wearable wireless patch that can be attached to human skin to conduct temperature measurements with high accuracy and faster respond time.

Moreover, disclosed wireless patch and charging station enable convenient charging of the disclosed wearable patch. The disclosed wearable patch includes three electrodes that can be electrically controlled to set the wearable patch to different states to accomplish the measurements and charging functions.

Furthermore, the present disclosure provides a system to effectively increase the wireless communication range of the disclosed wearable patch with external devices.

In one general aspect, the present invention relates to a wearable patch that includes a stretchable and permeable substrate, a temperature sensing unit mounted in the stretchable and permeable substrate, wherein the temperature sensing unit includes a first electrode configured to contact a user's skin; and a temperature sensor in thermal contact with the temperature sensor and configured to measure the user's skin temperature; a second electrode and a third electrode respectively attached to the stretchable and permeable substrate; a circuit substrate on the stretchable and permeable substrate, wherein the circuit substrate can include a control circuit electrically connected with the first electrode, the second electrode, and the third electrode; and a battery configured to supply power to the control circuit and the temperature sensor, wherein the first electrode, the second electrode, and the third electrode can form electric contacts with charging pins in a charging station when the wearable patch is docked in the charging station, wherein the control circuit can select a first mode for measuring a user's skin temperature and a second mode for charging the battery by the charging station via the first electrode, the second electrode, and the third electrode.

Implementations of the system may include one or more of the following. The wearable patch can further include a semiconductor chip mounted on the circuit substrate, wherein the semiconductor chip is configured to receive an electric signal from the temperature sensor in response to a temperature measurement of the user's skin. The wearable patch can further include an antenna mounted on the circuit substrate and in electric connection with the semiconductor chip, wherein the semiconductor chip can produce electric signals to enable the antenna to wirelessly exchange data with an external device, wherein the data include the temperature measurement of the user's skin. At least part of the control circuit can be fabricated in the semiconductor chip. The battery can be mounted on the stretchable and permeable substrate or the circuit substrate. The temperature sensing unit can include a conductive cup having a bottom portion mounted in a first opening in the stretchable and permeable substrate, wherein the conductive cup is thermally and electrically conductive, wherein the first electrode is in part formed by the conductive cup. The temperature sensor can be positioned inside and is in thermal conduction with the conductive cup. The temperature sensing unit can include: a thermally-conductive adhesive that fixes the temperature sensor to an inner surface of the conductive cup; and a thermally insulating material in a top portion of the conductive cup. The second electrode and the third electrode each can include an electrically conductive cup that is electrically connected to the control circuit in the circuit substrate, wherein the stretchable and permeable substrate comprises a second opening and a third opening in which the electrically conductive cups are respectively mounted. The electrically conductive cups can be respectively electrically connected with the control circuit. The first electrode can be assigned to function as one of a power supply pin, an input/output pin, or a ground pin in the control circuit. The second electrode and the second electrode can be assigned to function as two of the power supply pin, the input/output pin, or the ground pin in the control circuit that are not selected by the first electrode. The wearable patch can further include an adhesive layer between the stretchable and permeable substrate and the circuit substrate. The wearable patch can further include an elastic layer formed on the stretchable and permeable substrate, the circuit substrate, and the temperature sensing unit.

In another general aspect, the present invention relates to a charging and wireless boosting station that includes a charging circuit comprising charging pins that can form electric contacts with electrodes on a wearable patch that is docked in the charging station; a first antenna that can receive a first wireless signal from a wearable patch and to produce a reception electric signal; a controller that can control the charging circuit to charge the wearable patch or to control conversion and amplification of the first wireless signal transmitted by the wearable patch; an amplifier module that can amplify the reception electric signal to produce an amplified electric signal under the control of the controller; and a second antenna that can transmit a second wireless signal in response to the amplified electric signal.

Implementations of the system may include one or more of the following. The charging and wireless boosting station can further include a charging port comprising the charging pins and configured dock the wearable patch. The charging and wireless boosting station can further include a first battery in connection with the charging circuit, the first battery that can charge a second battery in the wearable patch when the wearable patch is docked in the charging station. The charging pins can include a ground pin, a power supply pin, and an input/output pin. The charging circuit can control voltages of the charging pins to set the wearable patch in at least the following modes: a first mode for measuring signals from a user, a second mode in which the wearable patch is shut off, or a third mode in which the wearable patch is being charged by the charging and wireless boosting station.

These and other aspects, their implementations and other features are described in detail in the drawings, the description and the claims.

### BRIEF DESCRIPTION OF THE DRAWINGS

FIG. 1 illustrates a wearable patch attached to a user's skin.

FIG. 2 is an exemplified cross-sectional view of a wearable patch comprising three electrodes enabling temperature measurement and battery charging in accordance with some embodiments of the present invention.

FIG. 3 is a detailed cross-sectional view of an exemplified temperature sensing unit in the wearable patch of FIG. 2.

FIG. 4 shows a station for charging and wireless boosting the disclosed wearable patch in accordance with some embodiments of the present invention.

FIG. 5 is a block circuit diagram showing circuits in the wearable patch and in the charging and wireless boosting station in accordance with some embodiments of the present invention.

FIG. 6 shows the states of the wearable patch in accordance with some embodiments of the present invention.

FIG. 7 shows the states of the charging and wireless boosting station in accordance with some embodiments of the present invention.

FIGS. 8A and 8B are respectively top and bottom perspective views of an exemplified wearable patch compatible with the present invention.

FIGS. 9A and 9B are respectively top and bottom perspective views of another exemplified wearable patch compatible with the present invention.

### DETAILED DESCRIPTION OF THE INVENTION

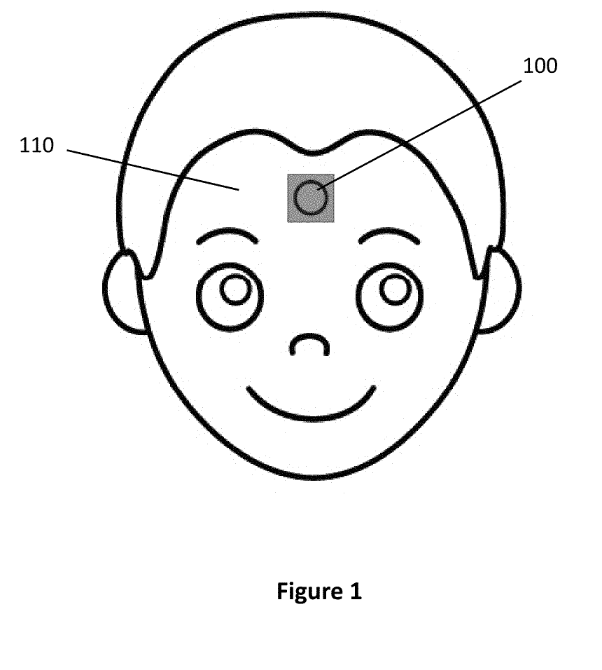

Referring to FIG. 1, a wearable patch 100 is attached to a user's skin 110 for measuring body vital signs. The wearable patch 100 can be placed on forehead, hand, wrist, arm, shoulder, waist, leg, foot, armpit, or other parts of the body. In the present disclosure, the term “wearable patch” can also be referred to as “wearable sticker” or “wearable tag”.

As discussed above, wearable electronic patches face several challenges: the user's skin 110 may interfere with their proper operations. For example, the wearable patch 100 may include an antenna for wireless communications with other devices. The antenna's communication range can be significantly reduced when an antenna is placed in contact with the user's skin 110.

The presently disclosed wearable patch and system aim to overcome the drawbacks in conventional wearable patches, and to provide highly stretchable, compliant, durable, breathable, and comfortable wearable electronic patches while performing more accurate and more responsive measurements and communication functions.

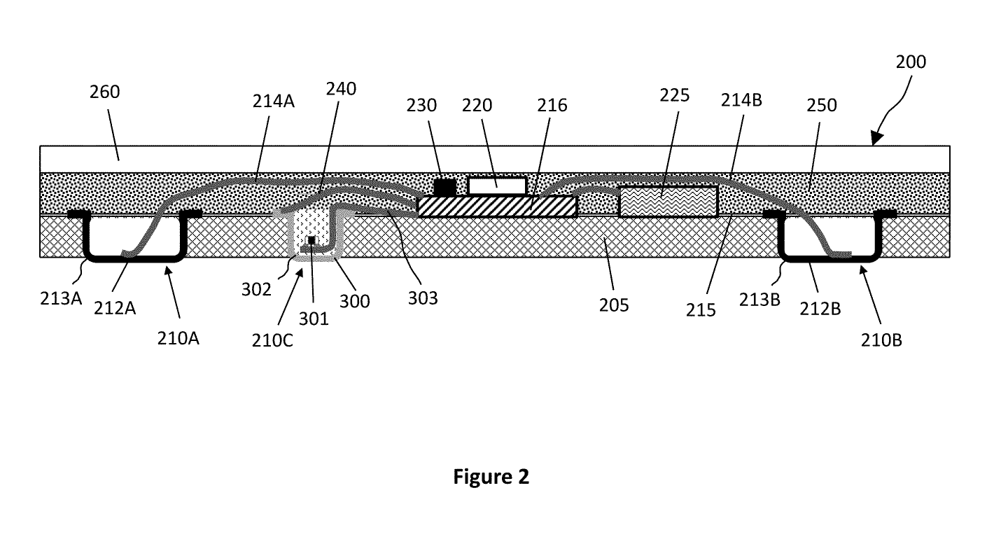

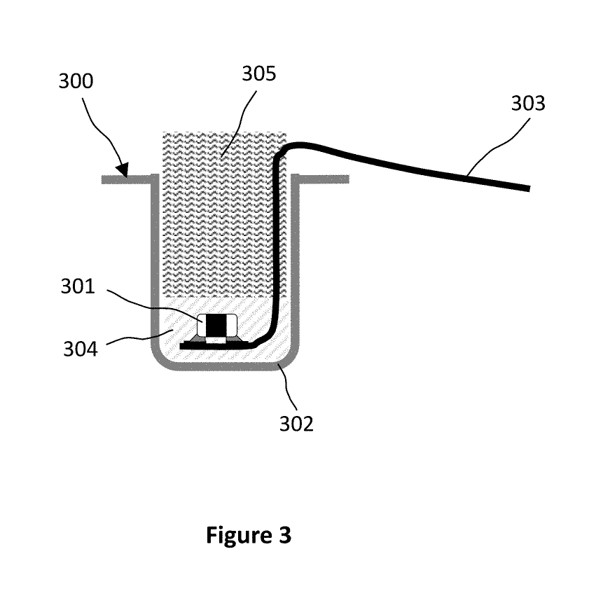

Referring to FIGS. 2 and 3, a wearable patch 200 includes a stretchable and permeable substrate 205 that include openings 210A, 210B, 210C. The stretchable and permeable substrate 205 can be made of soft foam materials such as EVA, PE, CR, PORON, EPD, SCF or fabric textile, to provide stretchability and breathability. A temperature sensing unit 300, with details shown in FIG. 3, is mounted in the opening 210C. Two electrodes 212A, 212B, respectively comprising electrically conductive cups 213A, 213B, are mounted in the openings 210A, 210B. A circuit substrate 216 and a battery 225 are bonded to the stretchable and permeable substrate 205 by an adhesive layer 215 pre-laminated on the stretchable and permeable substrate 205. A semiconductor chip 220 and an antenna 230 are mounted on the circuit substrate 216. The circuit substrate 216 includes an electric circuit therein and can for example be implemented with a printed circuit board.

The electrically conductive cups 213A, 213B in the electrodes 212A, 212B are respectively electrically connected to the electric circuit in the circuit substrate 216 by conductive lines 214A, 214B (e.g. flexible ribbons embedded with conductive circuits). In accordance with the present application, the electrodes 212A, 212B can also be implemented in other configurations such as conductive pins, conductive pads, conductive buttons, or conductive strips.

The temperature sensing unit 300, as described above, includes a conductive cup 302 having its bottom portion mounted into the large opening 210C and fixed to the stretchable and permeable substrate 205 by an adhesive. A temperature sensor 301 is electrically connected to the electric circuit in the circuit substrate 216 by a flexible conductive ribbon 303.

Referring to FIG. 3, the conductive cup 302 has its bottom portion mounted into the large opening 210 and fixed to the stretchable and permeable substrate 205 by an adhesive. The bottom portion of the conductive cup 302 protrudes out of the lower surface of the stretchable and permeable substrate 205. The lips of the conductive cup 302 near its top portion are fixedly attached or bonded to bonding pads (not shown) on the stretchable and permeable substrate 205 by soldering or with an adhesive. The conductive cup 302 is both thermally and electrically conductive. The conductive cup 302 can be made of a thermally conductive metallic or alloy material such as copper, stainless steel, ceramic or carbide composite materials.

A temperature sensor 301 is attached to an inner surface near the bottom of the conductive cup 302. The temperature sensor 301 can be implemented, for example, by a thermistor, a Resistor Temperature Detector, or a Thermocouple. The temperature sensor 301 is in thermal conduction with the conductive cup 302. When an outer surface of the bottom portion of the conductive cup 302 is in contact with a user's skin, the conductive cup 302 thus effectively transfers heat from a user's skin to the temperature sensor 301. A flexible conductive ribbon 303 is connected to the temperature sensor 301 in the conductive cup 302 and to the electric circuit in the stretchable and permeable substrate 205. The temperature sensor 301 can send an electric signal to the semiconductor chip 220 via the electric circuit in response to a measured temperature. The semiconductor chip 220 processes the electric signal and output another electrical signal which enables the antenna 230 to transmit a wireless signal carrying the measurement data to another external device such as a mobile phone or a computer (its wireless signals, as described below, can be boosted by a charging and wireless boosting station). The wireless signal can be based on using WiFi, Bluetooth, Near Field Communication (NFC), and other wireless standards. The battery 225 powers the semiconductor chip 220, the antenna 230, the first and the second electric circuits, and possibly the temperature sensor 301.

The temperature sensor 301 can be fixed to an inner surface at the bottom of the conductive cup 302 by a thermally-conductive adhesive 304, which allows effective heat transfer from the bottom of the conductive cup 302 to the temperature sensor 301. Examples of the thermally-conductive adhesive 304 can include electrically-insulative thermally-conductive epoxies and polymers. A thermally insulating material 305 filling the top portion of the conductive cup 302 fixes the thermally-conductive adhesive 304 at the bottom of the conductive cup 302 and reduces heat loss from the temperature sensor 301 to the elastic layer (described below) or the environment. The flexible conductive ribbon 303 can be bent and laid out along the wall the conductive cup 302.

Further details of the temperature sensing unit are disclosed in the commonly assigned co-pending U.S. patent application Ser. No. 15/224,121 “Wearable thermometer patch for accurate measurement of human skin temperature”, filed Jul. 29, 2016, the disclosure of which is incorporated herein by reference.

Referring back to FIG. 2, the conductive cup 302 in the temperature sensing unit 300 is electrically connected with the circuit substrate 216 by a conductive line 240, which in turn establishes electrical communication between the conductive cup 302 and the semiconductor chip 220. In usage, as described in more detail below, the conductive cup 302 can be used as a third electrode in addition to the electrodes 212A, 212B.

An elastic layer 250 is formed on the stretchable and permeable substrate 205, the circuit substrate 216, the temperature sensing unit 300, and the electrodes 212A, 212B. The elastic layer 250 can be formed by soft stretchable and permeable foam materials such as EVA, PE, CR, PORON, EPD, SCF, or fabric textile. A thin film 260 is formed on the elastic layer 250 for protection and cosmetic purposes.

In usage, an adhesive material formed on the lower surface of the stretchable and permeable substrate 205 is attached the user's skin, so that the bottom of the conductive cup 302 is in tight contact with a user's skin to accurately measure temperature and electric signals from the user's skin. The semiconductor chip 220 receives an electric signal from the temperature sensor 301 in response to a temperature measurement of the user's skin.

The semiconductor chip 220 processes the electric signal and output another electrical signal which enables the antenna 230 to transmit a wireless signal carrying the measurement data to another external device such as a mobile phone or a computer. The wireless signal can be based on using WiFi, Bluetooth, Near Field Communication (NFC), and other wireless standards. When the wearable patch 200 is worn by a user, the antenna 230 is separated from the user's skin by the circuit substrate 216 and the stretchable and permeable substrate 205, which minimizes the impact of the user's body on the transmissions of wireless signals by the antenna 230.

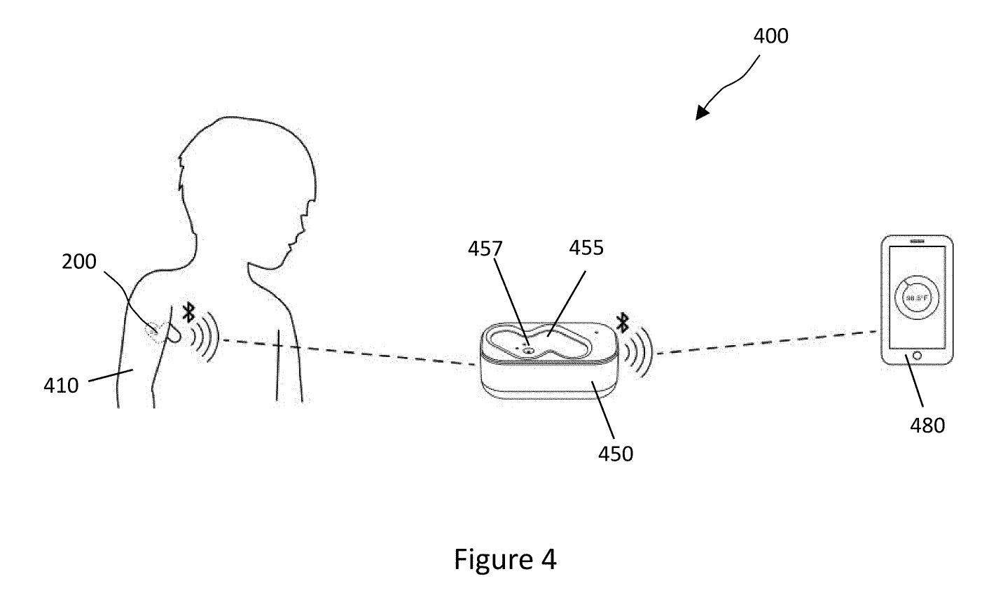

In some embodiments, referring to FIGS. 2 and 4, a wireless sensing system 400 includes the wearable patch 200 and a charging and wireless boosting station 450. The charging and wireless boosting station 450 includes a charging port 455 and charging pins 457 in the charging port 455. The wearable patch 200 can be worn by a user 410, or can be mounted on the charging and wireless boosting station 450 for charging its battery 225.

When the wearable patch 200 is mounted onto the charging and wireless boosting station 450, the bottom surfaces of the electrically conductive cups 213A, 213B in the electrodes 212A, 212B and the conductive cup 302 in temperature sensing unit 300 come to contact with the charging pins 457, which allows the charging and wireless boosting station 450 to charge the battery 225 in the wearable patch 200. More details about control and circuit for charging of the battery 225 are described in more detail below.

In operation, the wearable patch 200 is attached to the skin of the user 410. The antenna 230 in the wearable patch 200 can transmit a wireless signal carrying measurement, status, or control data to an external device 480 such as a mobile phone or a computer device. In some embodiments, the charging and wireless boosting station 450 can be positioned near the user 410 and the wearable patch 200 and to boost the wireless signals of the wearable device. The wireless signals transmitted by the wearable patch 200 is received and then boosted or amplified by the charging and wireless boosting station 450. Amplified wireless signals carrying the same measurement, status, or control data are transmitted by the charging and wireless boosting station 450 and received by the external device 480. The boosting of wireless signals from the wearable patch 200 enables much wider range of communication between the wearable patch 200 and the external device 480.

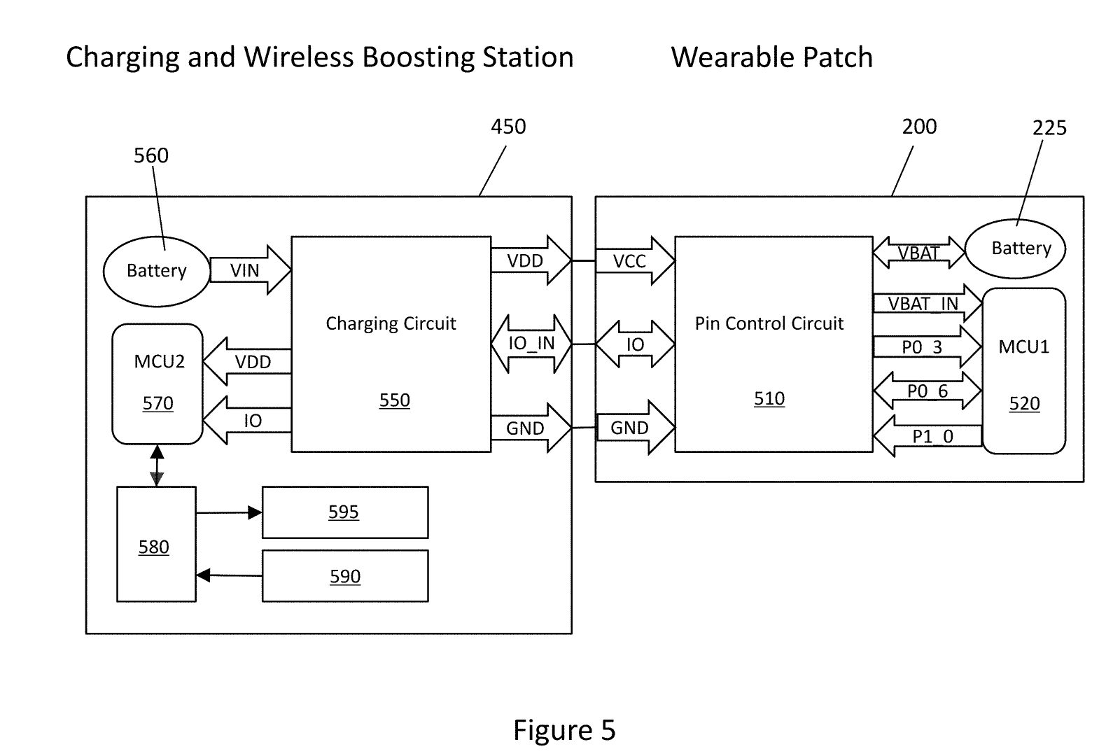

FIG. 5 is a block circuit diagram showing circuits in the wearable patch 200 and in the charging and wireless boosting station 450. A pin control circuit 510 is configured to control the switching between the sensing mode (i.e. measurement of user skin temperature) and the charging mode (when the wearable patch 200 is docked on the charging and wireless boosting station 450). The pin control circuit 510 can be part of the electric circuit in the circuit substrate 216 (FIG. 2) or can be fabricated in the semiconductor chip 220 (FIG. 2). The pin control circuit 510 powered by the battery 225 via pin VBAT in the wearable patch 200. The pin control circuit 510 can supply power to a microcontroller 520 (MCU1) via pins VBAT_IN and communicate with the microcontroller 520 via several other pins. The microcontroller 520 can be implemented in the semiconductor chip 220 (FIG. 2). In the charging and wireless boosting station 450, the charging circuit 550 is powered by a battery 560 via pin VIN, and controlled by a micro controller 570 (MCU2). The battery 560 has much larger capacity than the battery 225 in the wearable patch 200, and can be charged by a power outlet.

The pin control circuit 510 includes: a power supply pin VCC, an input/output pin IO, and a ground pin GND respectively corresponding to the electrodes 212A, 212B and the conductive cup 302 in temperature sensing unit 300 (FIG. 2). For example, the conductive cup 302 can be designed to perform as one of a power supply pin VCC, an input/output pin IO, or a ground pin GND in the pin control circuit 510. The electrodes 212A, 212B can be assigned to perform the functions of the rest of the two pins.

The charging circuit 550 includes: a power supply pin VDD, an input/output pin IO_IN, and a ground pin GND corresponding to the three charging pins 457 in the charging and wireless boosting station 450. When the wearable patch 200 is docked on the charging and wireless boosting station 450, the power supply pin VCC, IO, and GND pins of the pin control circuit 510 are respectively connected to the power supply pin VDD, IO_IN, and GND pins in the charging circuit 550.

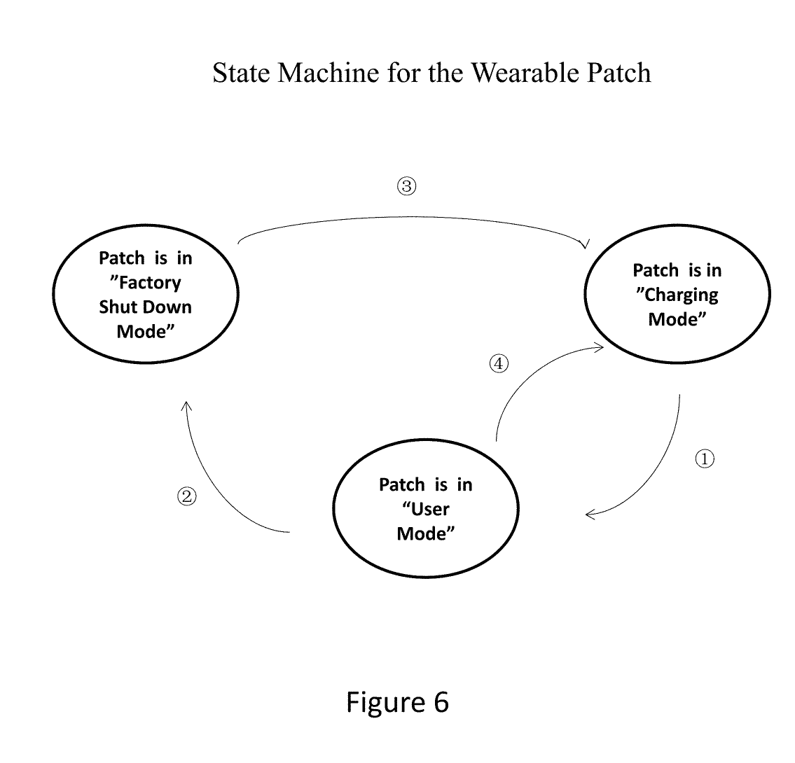

The different modes of the wearable patch and the corresponding logic states of the pin control circuit are summarized in Table I below and in FIG. 6. When both the power supply pin VCC and IO are low, the wearable patch is in a normal working mode (i.e. it can measure temperature and other signals in normal operation). When the wearable patch is docked on a station with VCC set low and IO set high, the wearable patch is turned off in a factor shut-down mode, which can preserve the wearable patch in a long shelf life. When VCC is set high and IO set low, the wearable patch is in a charging mode in which the battery 225 is being charged; its wireless function (e.g. Bluetooth) is turned off. When both VCC and IO are set high, the wearable patch is in a charging mode; its wireless function (e.g. Bluetooth) is turned off.

TABLE I
Pin Control and Modes in the Wearable Patch

| States | VCC | IO | Status |
|---|---|---|---|
| \textcircled{1} | 0 (input) | 0 (input) | Patch normal working |
| \textcircled{2} | 0 (input) | 1 (input) | Patch turned off |
| \textcircled{3} | 1 (input) | 0 (output) | Patch is activated, charging, |
| | | | wireless function off |
| \textcircled{4} | 1 (input) | 1 (output) | Patch charging and the |
| | | | wireless function off |

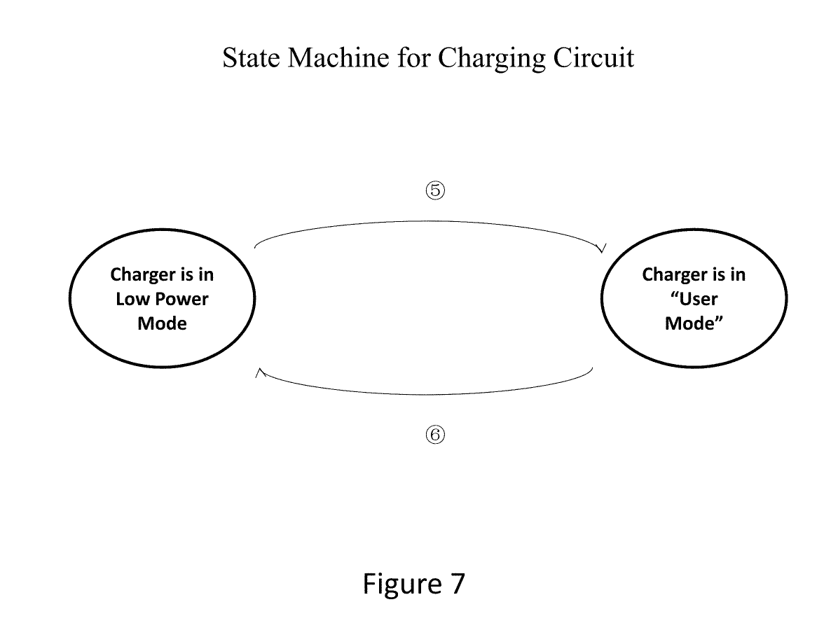

Referring to FIGS. 5 and 7, the charging circuit 550 can be in at least two modes: a user mode in which its charger is capable of charging a wearable patch 200; and a lower power mode in which the battery 560 does not have enough power to charge a wearable patch 200. The charging circuit 550 monitors the voltage level of the battery 560 and can prevents charging operation of the voltage level drops to below a threshold. In the user mode, the power in the battery 560 is enough for charging the battery 225 in the wearable patch 200. Via pins VDD and VCC, and the GND pins. Moreover, the charging circuit 550 can be in a separate wireless boosting mode in which the charging circuit 550 is not charging and the controller 570 controls an amplifier module 580 to boost wireless signals from the wearable patch 200.

In some embodiments, referring to FIGS. 4 and 5, an antenna 590 receives wireless signals (comprising measurement, status of control data) from the antenna 230 in the wearable patch 200 and outputs reception electric signals. The amplifier module 580 amplifies the reception electric signals under the control of the controller 570. The amplified reception electric signals are sent to another antenna 595 for a secondary wireless transmission with strong transmission power, which can be received by the external device 480.

In some embodiments, still referring to FIGS. 4 and 5, the charging and wireless boosting station 450 can include a down-converter that can convert reception RF signal from the antenna 590 to a base band reception signal. The controller 570 can process and analyze the base band reception signal before it sends another base band transmission signal to an up-converter (not shown) which converts it into an RF transmission signal such as BLE or WiFi, which is in turn transmitted by the antenna 595.

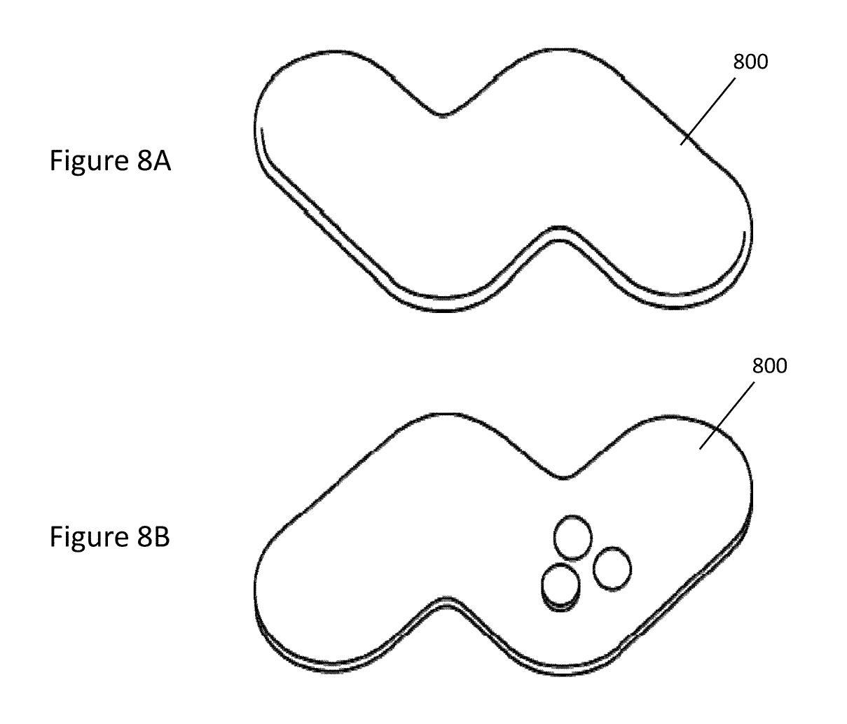

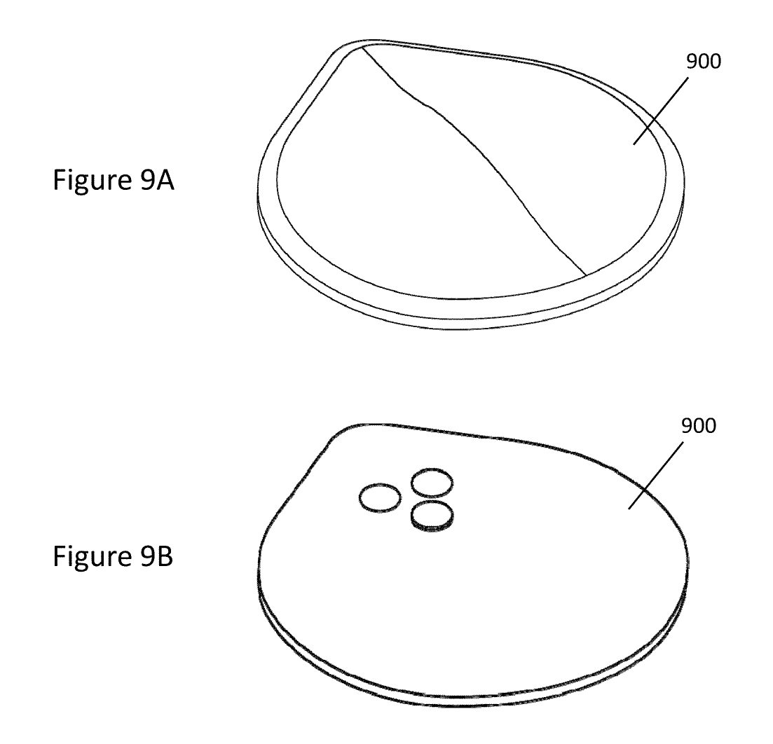

The disclosed wearable patch is compatible with a variety of exterior designs. FIGS. 8A and 8B shows an exemplified design of a wearable patch 800 that is compatible with the above described wearable patch 200, and the charging and wireless boosting station 450 in FIGS. 2 and 4. FIGS. 9A and 9B shows another exemplified design of a wearable patch 900 that is compatible with the above described wearable patch 200 in FIG. 2. The wearable patch in accordance with the present invention can be implemented in many other configurations. For example, the three electrodes can be positioned along a line or in a triangular fashion as shown in FIGS. 8A-9B.

The disclosed wearable patch can significantly enhance measurement accuracy and responsiveness, and reduce thermal noise. The temperature sensor is positioned very close to a user's skin. The temperature sensor is placed at the bottom of a thermally conductive cup and in good thermal conduction with the user's skin. The minimized thermal resistance between the temperature sensor and the user's skin reduces temperature measurement error and also decreases measurement response time. Moreover, the temperature sensor is secured fixed by an adhesive to the bottom of the thermally conductive cup such that the temperature sensor is not affected and detached by user's body movements, which improves durability of the wearable patch. Furthermore, the temperature sensor is thermally isolated with the ambient environment by a thermal insulating material in the top portion of the thermally conductive cup. The reduced thermal capacity helps further reduces background noise in the measurements of user's skin temperature and increase response rate of measurement. A layer of soft perforated polymer material under the flexible substrate minimizes heat conduction from the user's skin to the wearable patch, thus reducing the “cooling effect” of the user's skin by the wearable patch.

Another advantage of the disclosed wearable patch is that it is stretchable, compliant, durable, and comfortable to wear by users. The disclosed wearable thermometer patch includes a flexible substrate covered and protected by an elastic layer that increases the flexibility and stretchability.

Yet another advantage of the disclosed wearable patch is that it can significantly increase wireless communication range by placing the antenna on the upper surface of the circuit substrate. The thickness of the substrate as well as the height of the thermally conductive cup can be selected to allow enough distance between the antenna and the user's skin to minimize interference of user's body to the wireless transmission signals.

The disclosed wearable patches can also include electronic components such as the semiconductor chips, resistors, capacitors, inductors, diodes (including for example photo sensitive and light emitting types), other types of sensors, transistors, amplifiers. The sensors can also measure temperature, acceleration and movements, force, strain and stress, and chemical or biological substances. The semiconductor chips can perform communications, logic, signal or data processing, control, calibration, status report, diagnostics, and other functions.

While this document contains many specifics, these should not be construed as limitations on the scope of an invention that is claimed or of what may be claimed, but rather as descriptions of features specific to particular embodiments. Certain features that are described in this document in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination.

Only a few examples and implementations are described. Other implementations, variations, modifications and enhancements to the described examples and implementations may be made without deviating from the spirit of the present invention.

What is claimed is:

1. A wearable patch, comprising:
a stretchable and permeable substrate;
a temperature sensing unit mounted in the stretchable and permeable substrate, wherein the temperature sensing unit comprises:
a first electrode configured to contact a user's skin; and
a temperature sensor in thermal contact with the temperature sensor and configured to measure the user's skin temperature;
a second electrode and a third electrode respectively attached to the stretchable and permeable substrate;
a circuit substrate on the stretchable and permeable substrate, wherein the circuit substrate comprises a control circuit electrically connected with the first electrode, the second electrode, and the third electrode; and
a battery configured to supply power to the control circuit and the temperature sensor, wherein the first electrode, the second electrode, and the third electrode are configured to form electric contacts with charging pins in a charging station when the wearable patch is docked in the charging station, wherein the control circuit is configured to select a first mode for measuring a user's skin temperature and a second mode for charging the battery by the charging station via the first electrode, the second electrode, and the third electrode.

2. The wearable patch of claim 1, further comprising:
a semiconductor chip mounted on the circuit substrate, wherein the semiconductor chip is configured to receive an electric signal from the temperature sensor in response to a temperature measurement of the user's skin.

3. The wearable patch of claim 2, further comprising:
an antenna mounted on the circuit substrate and in electric connection with the semiconductor chip, wherein the semiconductor chip is configured to produce electric signals to enable the antenna to wirelessly exchange data with an external device, wherein the data include the temperature measurement of the user's skin.

4. The wearable patch of claim 2, wherein at least part of the control circuit is fabricated in the semiconductor chip.

5. The wearable patch of claim 1, wherein the battery is mounted on the stretchable and permeable substrate or the circuit substrate.

6. The wearable patch of claim 1, wherein the temperature sensing unit includes a conductive cup having a bottom portion mounted in a first opening in the stretchable and permeable substrate, wherein the conductive cup is thermally and electrically conductive, wherein the first electrode is in part formed by the conductive cup.

7. The wearable patch of claim 6, wherein the temperature sensor is positioned inside and is in thermal conduction with the conductive cup.

8. The wearable patch of claim 7, wherein the temperature sensing unit comprises:
a thermally-conductive adhesive that fixes the temperature sensor to an inner surface of the conductive cup; and
a thermally insulating material in a top portion of the conductive cup.

9. The wearable patch of claim 1, wherein the second electrode and the third electrode each comprising an electrically conductive cup that is electrically connected to the control circuit in the circuit substrate, wherein the stretchable and permeable substrate comprises a second opening and a third opening in which the electrically conductive cups are respectively mounted.

10. The wearable patch of claim 9, wherein the electrically conductive cups are respectively electrically connected with the control circuit.

11. The wearable patch of claim 1, wherein the first electrode is assigned to function as one of a power supply pin, an input/output pin, or a ground pin in the control circuit.

12. The wearable patch of claim 11, wherein the second electrode and the second electrode are assigned to function as two of the power supply pin, the input/output pin, or the ground pin in the control circuit that are not selected by the first electrode.

13. The wearable patch of claim 1, further comprising:
an adhesive layer between the stretchable and permeable substrate and the circuit substrate.

14. The wearable patch of claim 1, further comprising:
an elastic layer formed on the stretchable and permeable substrate, the circuit substrate, and the temperature sensing unit.

15. A charging and wireless boosting station, comprising:
a charging circuit comprising charging pins configured to form electric contacts with electrodes on a wearable patch that is docked in the charging station;
a first antenna configured to receive a first wireless signal from a wearable patch and to produce a reception electric signal;
a controller configured to control the charging circuit to charge the wearable patch or to control conversion and amplification of the first wireless signal transmitted by the wearable patch;
an amplifier module configured to amplify the reception electric signal to produce an amplified electric signal under the control of the controller; and
a second antenna configured to transmit a second wireless signal in response to the amplified electric signal.

16. The charging and wireless boosting station of claim 15, further comprising:
a charging port comprising the charging pins and configured dock the wearable patch.

17. The charging and wireless boosting station of claim 15, further comprising:
a first battery in connection with the charging circuit, the first battery configured to charge a second battery in the wearable patch when the wearable patch is docked in the charging station.

18. The charging and wireless boosting station of claim 15, wherein the charging pins comprise a ground pin, a power supply pin, and an input/output pin.

19. The charging and wireless boosting station of claim 15, wherein the charging circuit is configured to control voltages of the charging pins to set the wearable patch in at least the following modes: a first mode for measuring signals from a user, a second mode in which the wearable patch is shut off, or a third mode in which the wearable patch is being charged by the charging and wireless boosting station.
